(19) United States
(12) Patent Application Publication
(10) Pub. No.: US 2024/0198021 A1
(43) Pub. Date: Jun. 20, 2024

(54) PIPELINE STRUCTURE CONFIGURED TO PREVENT BACKFLOW OF CONDENSED WATER AND BREATHING MACHINE

(71) Applicant: RESVENT MEDICAL TECHNOLOGY CO., LTD, SHENZHEN (CN)

(72) Inventors: KAI QU, SHENZHEN (CN); RONGRONG ZHU, SHENZHEN (CN)

(21) Appl. No.: 18/493,785

(22) Filed: Oct. 24, 2023

(30) Foreign Application Priority Data
Dec. 16, 2022 (CN) 202223387814.4

(51) Int. Cl. A61M 16/00; A61M 16/10; F16L 41/02

(52) U.S. Cl. CPC A61M 16/0003; A61M 16/1075; F16L 41/02

(57) ABSTRACT

The present disclosure provides a pipeline structure configured to prevent backflow of condensed water and a breathing machine. The pipeline structure includes a first branch, a second branch, a third branch, and a flow guide component. A first end of the first branch is a first air port. A second end of the first branch is a first communicating port. A first end of the second branch is a second air port. A second end of the second branch is communicated with the first communicating port. A first end of the third branch is a third air port. A second end of the third branch is communicated with the first communicating port. The flow guide component directly faces the first communicating port. The flow guide component is configured to guide an air of the first branch to the third branch and discharge the air through the third air port.

### TECHNICAL FIELD

The present disclosure relates to a technical field of medical instruments, and in particular to a pipeline structure configured to prevent backflow of condensed water and a breathing machine.

### BACKGROUND

Breathing machines are vital medical devices capable of preventing and treating respiratory failure, reducing complications, saving and prolonging life of patients. In modern clinical medicine, the breathing machines serve as an effective machine capable of manually replacing an autonomous ventilation function, are generally used for respiratory failure caused by various reasons, anesthesia respiration management during large operations, respiratory support treatment, and first-aid resuscitation, so that the breathing machines occupy a very important position in a field of modern medicine. It is generally necessary to connect an external humidifier when using a breathing machine. An air direction is changed during a breathing process of a patient, and when the patient inhales, the air is sent out through an air suction branch of the breathing machine, passes through the humidifier, and reaches the patient. In the process, the humidifier heats and humidifies the air. When the patient exhales, the breathing machine stops supplying the air. Since an expiration pressure exists in an expiration branch or a leakage hole, one part of the air exhaled by the patient enters the air suction branch through the humidifier, the air entering the air suction branch contacts a pipe wall with a lower temperature, water vapor in the air flow is condensed into condensed water on the pipe wall, and the condensed water is blown into the breathing machine by the air, so that components in the breathing machine are damaged under long-term erosion of the condensed water, which affects service life of the breathing machine.

According to a current technical scheme, a diaphragm type check valve is adopted to prevent the condensed water from entering the components in the breathing machine. The diaphragm type check valve only allows the air to pass through in one direction. When the patient inhales, the air flows from an interior of the breathing machine to outside of the breathing machine, a diaphragm is blown to open the check valve so the air passes through the check valve. When the patient exhales, the diaphragm is closed, and at this time, the air carrying the water vapor and the condensed water is blocked by the diaphragm and is unable to be blown into the breathing machine.

Problems existing in the prior art are as follow.

Firstly, for a breathing machine using a single pipeline, the pipeline is closed when the patient exhales, and the air only flows out from discharge holes of a mask and the pipeline, so that an expiration pressure is high, and an expiration process is not smooth. Secondly, the diaphragm made of silicone may age, damage, stick together, etc., after long-time use, so that use risk of the machine is increased, and the cost is high.

### SUMMARY

In the prior art, a conventional breathing machine generally adopts a diaphragm type check valve to prevents condensed water from entering an interior thereof, leading to problems such as unsmooth exhalation process of a patient, high use risk, and high cost. In order to solve above problems, the present disclosure provides a pipeline structure configured to prevent backflow of condensed water and a breathing machine.

The present disclosure provides the pipeline structure configured to prevent backflow of condensed water. The pipeline structure comprises a first branch, a second branch, a third branch, and a flow guide component. A first end of the first branch is a first air port. The first air port is configured to connect to a human body. A second end of the first branch is a first communicating port. A first end of the second branch is a second air port. The second air port is configured to connect to a breathing machine body. A second end of the second branch is communicated with the first communicating port. A first end of the third branch is a third air port. A second end of the third branch is communicated with the first communicating port. The flow guide component directly faces the first communicating port. The flow guide component is configured to guide an air of the first branch to the third branch and discharge the air through the third air port.

Furthermore, the flow guide component defines a flow guide cavity. The flow guide cavity includes an air inlet cavity port. The air inlet cavity port faces the first communicating port. An area of the air inlet cavity port is greater than an area of the first communicating port.

Furthermore, the second branch includes a second communicating port. The second communicating port is configured to connect the first communicating port. The second branch is bent in a zigzag shape from the second air port to the second communicating port.

Furthermore, the second branch is bent in an upward zigzag shape from the second air port to the second communicating port. The third branch is disposed below the flow guide component.

Furthermore, the flow guide cavity further includes an air outlet cavity port. The third branch is connected to the air outlet cavity port. An included angle between the third branch and the flow guide cavity is an acute angle.

Furthermore, the pipeline structure further includes a pipeline main body. The first branch, the second branch, the third branch, and the flow guide component are disposed in the pipeline main body. The pipeline main body includes a first housing and a second housing. The first housing and the second housing are assembled to form the pipeline main body.

Furthermore, the first housing includes a protruding cavity. The protruding cavity includes the first communicating port, the second air port, and an opening. The second housing is configured to seal the opening. The third branch is disposed in the second housing. The second branch and the flow guide component are disposed on the second housing.

Furthermore, the flow guide component is a plate body with a length. One side, opposite to an inner wall of the protruding cavity, of the flow guide component is the second branch. One side, opposite to the second housing, of the flow guide component is the flow guide cavity. The flow guide cavity includes the air inlet cavity port. The air inlet cavity port faces the first communicating port. The area of the air inlet cavity port is greater than the area of the first communicating port.

Furthermore, the flow guide cavity further includes the air outlet cavity port. The air outlet cavity port is defined on the second housing and is connected to the third branch.

The breathing machine includes the pipeline structure and a humidifier. The humidifier is connected to the first air port.

Compared with the prior art, the pipeline structure enables the dry air to flow into the second branch from the second air port. Then, the dry air flows into the first branch from the second branch, and finally flows out of the first air port. When humid air enters the first branch from the first air port and enters a common cavity, the humid air is guided to the third branch by the flow guide component and is finally discharged from the third branch, which prevents the humid air exhaled by the patient or the condensed water formed in the pipeline structure from flowing back from the first air port to an interior of the breathing machine body, thus preventing components inside the breathing machine body from erosion. Moreover, the air is directly discharged out of the pipeline structure from the third air port. The third air port greatly reduces an air inlet pressure at the first air port, in other words, an expiration pressure of the patient is reduced, making an expiration process of the patient smooth, and improving patient experience.

### BRIEF DESCRIPTION OF DRAWINGS

In order to more clearly illustrate technical solutions in embodiments of the present disclosure, drawings that need to be used in the embodiments or description of prior art are briefly described below. Obviously, the drawings in the following description are merely some embodiments of the present disclosure. For a person of ordinary skill in the art, other drawings are obtained according to the drawings without involving any inventive effort.

FIG. 1 is a perspective structural schematic diagram of a pipeline structure the present disclosure.

FIG. 2 is a cross-sectional structural schematic diagram of the pipeline structure of the present disclosure.

FIG. 3 is an exploded perspective structural schematic diagram of the pipeline structure of the present disclosure.

FIG. 4 is an exploded side structural schematic diagram of the pipeline structure of the present disclosure.

FIG. 5 is a schematic diagram of the pipeline structure of the present disclosure shown in an air feeding state.

FIG. 6 is a schematic diagram of the pipeline structure of the present disclosure shown in an air discharging state.

Reference numbers in the drawings: 1. first branch; 11. first air port; 12. first communicating port; 2. second branch; 21. second air port; 22. second communicating port; 3. third branch; 31. third air port; 4. flow guide component; 41. flow guide cavity; 411. air inlet cavity port; 412. air outlet cavity port; 5. first housing; 51. protruding cavity; 52. opening; 6. second housing; 61. sealing plate.

### DETAILED DESCRIPTION

In order to make technical problems to be solved, solutions, and beneficial effects clearer, following is a further detailed explanation of the present disclosure in conjunction with accompanying drawings and implementation examples. It should be understood that the specific embodiments described here are only intended to explain the present disclosure and are not intended to limit the present disclosure.

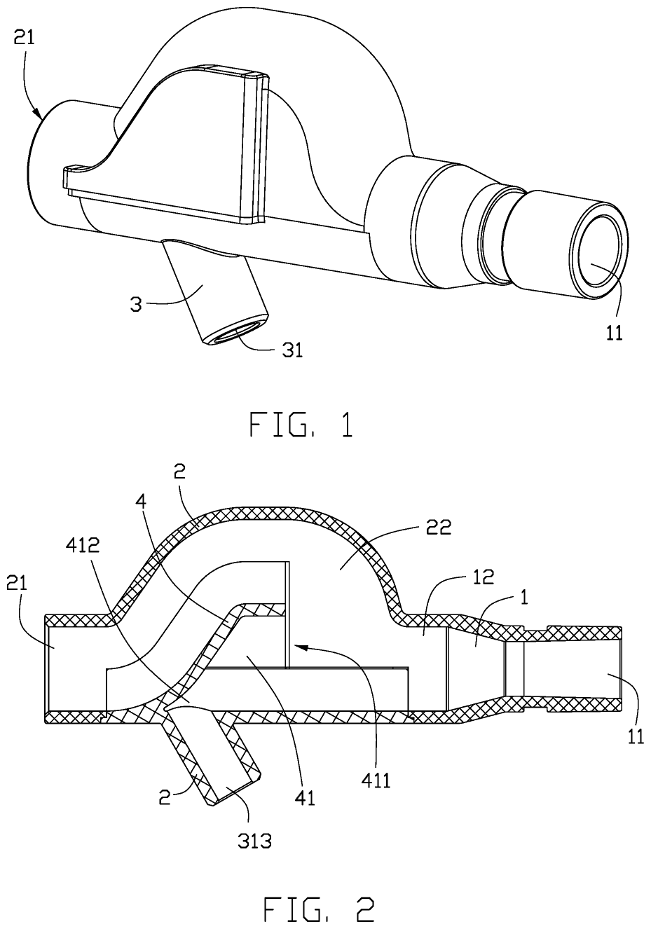

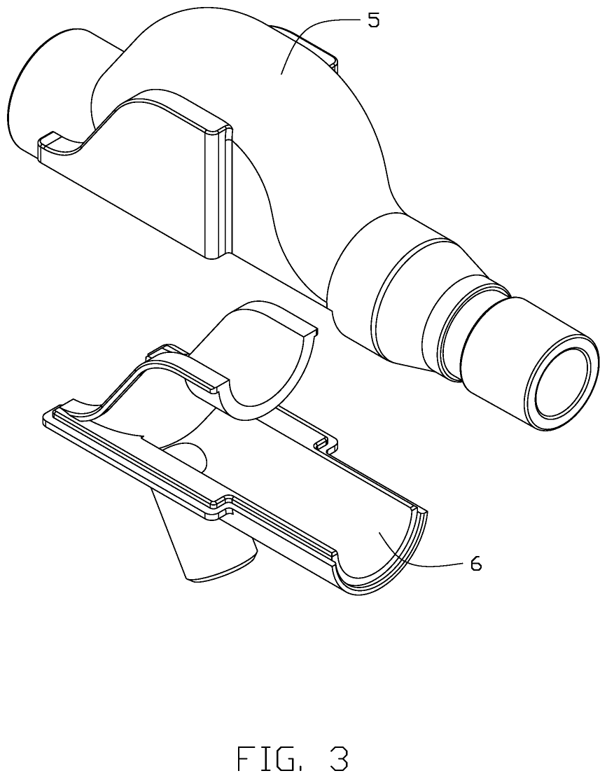

The present disclosure provides a pipeline structure configured to prevent backflow of condensed water. As shown in FIGS. 1-2, the pipeline structure includes: a first branch 1, a second branch 2, a third branch 3, and a flow guide component 4. The first branch 1, the second branch 2, and the third branch 3 are connected to a same point position to form a three-way flow path. Specifically, the first branch 1 includes two opening ends. A first end of the first branch 1 is a first air port 11. A second end of the first branch 1 is a first communicating port 12. The first air port 11 is mainly configured to connect to a human body, which allows dry air to flow out and allows humid air to enter an interior of the pipeline structure. The second branch 2 also includes two opening ends. A first opening end of the second branch 2 is a second air port 21. A second opening end of the second branch 2 is a second communicating port 22. The second air port 21 is mainly configured to connect to a breathing machine for the dry air to enter the pipeline structure. The second communicating port 22 is configured to connect to the first communicating port 12. The third branch 3 also includes two opening ends. A first opening end of the third branch 3 is configured to communicate with the first communicating port 12. A second opening end of the third branch 3 is a third air port 31. The third air port 31 is mainly configured to allow the humid air to flow out. The first branch 1, the second branch 2, and the third branch 3 form a common cavity at a joint thereof, and the dry air or the humid air flows through the common cavity. The flow guide component 4 is disposed at the common cavity. The flow guide component 4 is configured to guide the humid air entering the first branch 1 to the third branch 3, so that the humid air is finally discharged out of the pipeline structure from the third air port 31. By such arrangements, the dry air is allowed to flow into the second branch 2 from the second air port 21. Then, the dry air flows into the first branch 1 from the second branch 2, and finally flows out of the first air port 11. When the humid air enters the first branch 1 from the first air port 11 and enters the common cavity, the humid air is guided to the third branch 3 by the flow guide component 4 and is finally discharged from the third branch 3, which prevents the humid air exhaled by the patient or the condensed water formed in the pipeline structure from flowing back into an interior of the breathing machine body from the first air port, thus preventing components inside the breathing machine body from erosion. Moreover, the humid air is directly discharged out of the pipeline structure from the third air port 31. The third air port 31 greatly reduces air inlet pressure at the first air port 11, in other words, an expiration pressure of the patient is reduced, making an expiration process of the patient smooth, and improving patient experience.

Furthermore, the flow guide component 4 is disposed in the common cavity formed by the first branch 1, the second branch 2, and the third branch 3. The flow guide component 4 includes a flow guide cavity 41. The flow guide cavity 41 includes two opening ends. A first opening end of the flow guide cavity 41 is large in size and is an air inlet cavity port 411. A second opening end of the flow guide cavity 41 is small in size and is an air outlet cavity port 412. The air outlet cavity port 412 of the flow guide cavity 41 is communicated with the third branch 3. The air inlet cavity port 411 of the flow guide cavity 41 is located at the common cavity and faces the first communicating port 12. An air inlet area of the air inlet cavity port 411 is greater than an air outlet area of the first communicating port 12, so that air discharged from the first communicating port 12 directly enters the air inlet cavity port 411, and then the air flows into the third branch 3 through the flow guide cavity 41 to discharge out of the pipeline structure.

Furthermore, in one specific embodiment, an axial direction of the first air port 11 is parallel to an axial direction of the second air port 21, and the first air port 11 and the second air port 21 are basically coaxially disposed. The first branch 1 is disposed in a form of a straight pipe, the second branch 2 is disposed in a form of a bent pipe in a zigzag shape. The second communicating port 22 of the second branch 2 is close to the first communicating port 12 and is communicated with the first communicating port 12. The second branch 2 is disposed to be the bent pipe in the zigzag shape, thereby increasing a pressure of the air flowing from the first branch 1 to the second branch 2, avoiding the warm and humid air from entering the second branch 2 to a certain extent when a user exhales, and further avoiding reflux of the warm and humid air into the interior of the breathing machine body, which effectively reduces erosion on components by water vapor, reduces failure rate of the breathing machine body, and improves service life of the breathing machine body.

Furthermore, position relationships of the branches are further limited in the present disclosure. The second branch 2 is bent in an upward zigzag shape from the second air port 21 to the second communicating port 22, the flow guide component 4 is disposed below the second branch 2, and the third branch 3 is disposed below the flow guide component 4, so that when the patient exhales, the condensed water formed by the warm and humid air contacting a pipe wall of the pipeline structure having a lower temperature flows into the third branch 3 from the flow guide cavity 41 and finally flows out.

An included angle between the third branch 3 and the flow guide cavity 41 is an acute angle, which guarantees that when the patient inhales, the air flows in from the first air port 11 and flows along the second branch 2. Further, the included angle between the third branch 3 and the flow guide cavity 41 is the acute angle, so that the air continuously flows in the flow guide cavity 41 in a vortex shape, which guarantees that the patient normally uses the breathing machine. In this way, there is no need to dispose an extra check valve, thereby minimizing design and manufacturing costs.

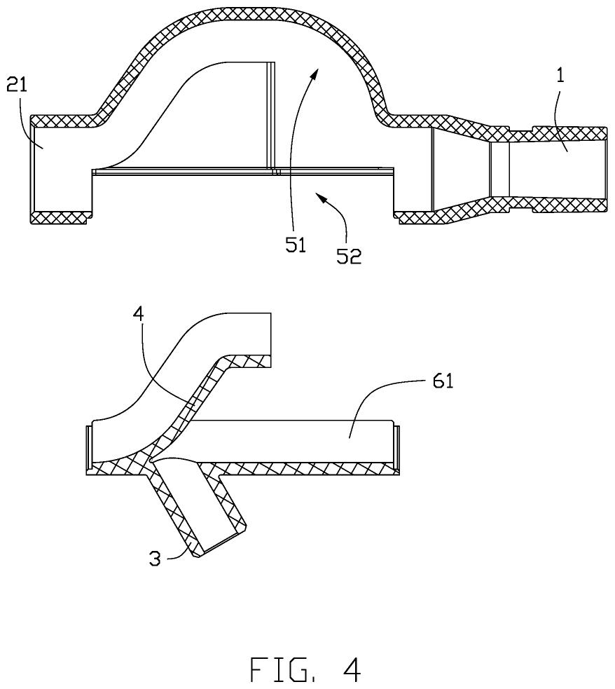

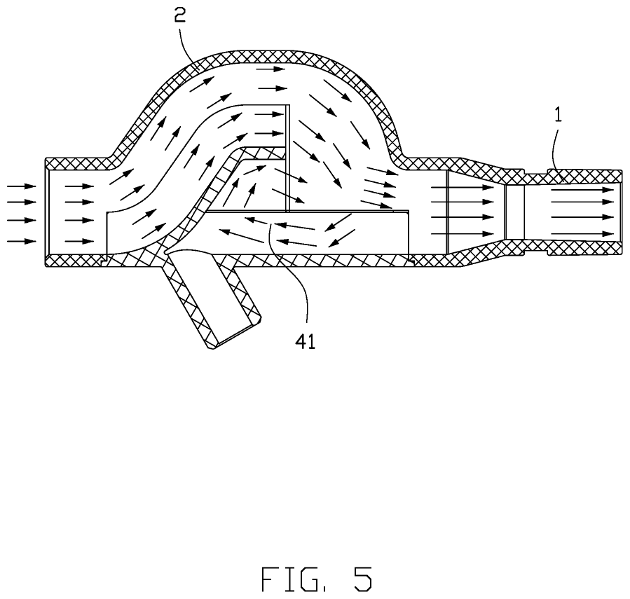

As shown in FIGS. 3-4, the pipeline structure further includes a pipeline main body. The first branch 1, the second branch 2, the third branch 3, and the flow guide component 4 are disposed in the pipeline main body. The pipeline main body is designed in an integrated mode or in a split mode. In a specific embodiment provided by the present disclosure, the pipeline main body is designed in a split mode. The pipeline main body includes a first housing 5 and a second housing 6. The first housing 5 and the second housing 6 are positioned through grooves and protrusions. The first housing 5 is fixed connected to and sealed with the second housing 6 through adhesive materials or ultrasonic welding. The first housing 5 is assembled with the second housing 6, which greatly reduces manufacturing and production difficulty, and reduces production costs.

Specifically, the second air port 21 is defined on a left end of the first housing 5. The first communicating port 12 is defined on a right end of the first housing 5. A pipe body of the first branch 1 is directly connected to the first communicating port 12. a protruding cavity 51 is formed in a middle of the first housing 5. The protruding cavity 51 is extended upwards. Two ends of the protruding cavity 51 are respectively connected to the second air port 21 and the first communicating port 12 and the protruding cavity form an upward arched arc-shaped flow channel from the second air port 21 to the first communicating port 12. A cross section of the flow channel is semicircular. An opening 52 is defined on a lower section of the protruding cavity 51. The second housing 6 is fixed at the opening 52 to seal the protruding cavity 51. The second housing 6 includes a sealing plate 61. A shape of the sealing plate 61 is matched with a shape of the opening 52, so that the sealing plate 61 is engaged with the opening 52 to realize sealing and fixing of the first housing and the second housing. The sealing plate 61 in the present disclosure is designed to be a straight pipe with a semicircular cross section so as to be matched with a shape of the pipeline structure.

In a specific embodiment, the flow guide component 4 is disposed on the sealing plate 61. The flow guide component 4 is a plate body with a certain length. A first end of flow guide component 4 connected to the sealing plate 61 is extended away from the sealing plate 6, and the flow guide component 4 extends in a bent shape similar to “S”. A cross section of the flow guide component 4 is a semicircular. An upper concave surface of the flow guide component 4 faces an inner wall of the protruding cavity 51, and the upper concave surface of the flow guide component 4 and the inner wall of the protruding cavity 51 together form the second branch 2. A lower convex surface of the flow guide component 4 faces the sealing plate 61 of the second housing 6, and the lower convex surface of the flow guide component 4 and the sealing plate 61 together form the flow guide cavity 41. The air outlet cavity port 412 of the flow guide cavity 41 is defined by the sealing plate 61, and the air outlet cavity port 412 is close to the flow guide component 4. An extending end of the flow guide component 4 distal from the sealing plate 61 faces he first communicating port 12. An extending end of the flow guide component 4 exceeds an upper wall of the first communicating port 12, which guarantees that the area of the air inlet cavity port 411 is greater than the area of the first communicating port 12. The third branch 3 is served as a drain pipe connected to outside, and the third branch 3 is directly communicated with the air outlet cavity port 412.

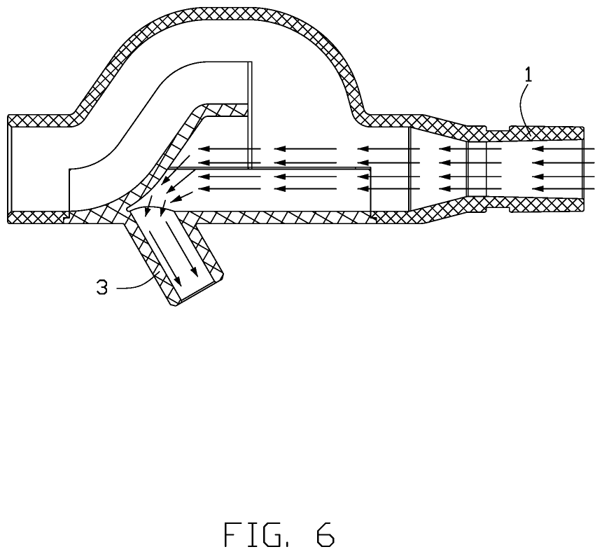

Referring to FIGS. 5-6, a working principle of the pipeline structure is as follow.

When the patient inhales, the air flows in from the second air port 21 and flows along the second branch 2. The third branch 3 is located below the flow guide component 4, and the included angle between the direction of the third branch 3 and the flow guide cavity 41 is the acute angle, so that the air continuously flows in the flow guide cavity 41 in the vortex shape. At this time, the air cannot be discharged along the third branch 3, but is continuously flows into the first branch 1, and finally reaches the human body through the first air port 11.

When the patient exhales, the air enters from the first air port 11 after being heated and humidified by a humidifier, and the air flows out of the first branch 1 from the first communicating port 12 and directly enters the flow guide cavity 41. Since the temperature of the pipe wall is lower than a temperature of the humid air, the water vapor in the humid air is converted into the condensed water on the pipe wall (i.e., an inner wall of the flow guide cavity 41), and under a combined action of reverse air and the flow guide cavity 41, the condensed water is blown to the air outlet cavity port 412 and is discharged through the third branch 3. Since the air inlet cavity port 411 of the flow guide cavity 41 faces the first communicating port 12, the area of the air inlet cavity port 411 is greater than the area of the first communicating port 12, and the extending end of the flow guide component 4 is higher than an upper end edge of the first communicating port 12 (i.e., the upper wall the first communicating port 12), so that the air reversely flowing into the pipeline main body does not flow through the second branch 2, and the condensed water cannot enter the breathing machine body through the second branch 2, which effectively reduces the erosion of the water vapor on the components, reduces the failure rate of the breathing machine body, and improves the service life of the breathing machine body.

Furthermore, the present disclosure further discloses the breathing machine. The breathing machine includes the pipeline structure. The breathing machine includes the breathing machine body and the humidifier. The breathing machine body is connected to the second air port 21. The humidifier is connected to the first air port 11.

The above are only preferred embodiments of the present disclosure, and are not intended to limit the present disclosure. Any modification, equivalent replacement, improvement, etc. made within spirit and principle of the present disclosure should be included within a scope of protection of the present disclosure.

What is claimed is:

1. A pipeline structure configured to prevent backflow of condensed water, comprising:
a first branch;
a second branch;
a third branch; and
a flow guide component;
wherein a first end of the first branch is a first air port, the first air port is configured to connect to a human body, and a second end of the first branch is a first communicating port;
a first end of the second branch is a second air port, the second air port is configured to connect to a breathing machine body, and a second end of the second branch is communicated with the first communicating port;
a first end of the third branch is a third air port, and a second end of the third branch is communicated with the first communicating port; and
the flow guide component directly faces the first communicating port, and the flow guide component is configured to guide an air of the first branch to the third branch and discharge the air through the third air port.

2. The pipeline structure according to claim 1, wherein the flow guide component defines a flow guide cavity, the flow guide cavity comprises an air inlet cavity port, the air inlet cavity port faces the first communicating port, and an area of the air inlet cavity port is greater than an area of the first communicating port.

3. The pipeline structure according to claim 2, wherein the second branch comprises a second communicating port, the second communicating port is configured to connect to the first communicating port, and the second branch is bent in a zigzag shape from the second air port to the second communicating port.

4. The pipeline structure according to claim 3, wherein the second branch is bent in an upward zigzag shape from the second air port to the second communicating port, and the third branch is disposed below the flow guide component.

5. The pipeline structure according to claim 4, wherein the flow guide cavity further comprises an air outlet cavity port, the third branch is connected to the air outlet cavity port, and an included angle between the third branch and the flow guide cavity is an acute angle.

6. The pipeline structure according to claim 1, wherein the pipeline structure further comprises a pipeline main body; the first branch, the second branch, the third branch, and the flow guide component are disposed in the pipeline main body, the pipeline main body comprises a first housing and a second housing, and the first housing and the second housing are assembled to form the pipeline main body.

7. The pipeline structure according to claim 6, wherein the first housing comprises a protruding cavity; the protruding cavity defines the first communicating port, the second air port, and an opening; the second housing is configured to seal the opening, the third branch is disposed in the second housing, and the second branch and the flow guide component are disposed on the second housing.

8. The pipeline structure according to claim 7, wherein the flow guide component is a plate body with a certain length; one side, opposite to an inner wall of the protruding cavity, of the flow guide component is the second branch; one side, opposite to the second housing, of the flow guide component is the flow guide cavity; the flow guide cavity comprises the air inlet cavity port, the air inlet cavity port faces the first communicating port, and an area of the air inlet cavity port is greater than an area of the first communicating port.

9. The pipeline structure according to claim 8, wherein the flow guide cavity comprises an air outlet cavity port, and the air outlet cavity port is defined on the second housing and is connected to the third branch.

10. A breathing machine, comprising the pipeline structure according to claim 1, and a humidifier; wherein the humidifier is connected to the first air port.
